(19) United States
(12) Patent Application Publication
(10) Pub. No.: US 2015/0251157 A1
(43) Pub. Date: Sep. 10, 2015

(54) SUPERHYDROXYLATED ADSORBENTS AND USES THEREOF

(71) Applicants: AFFIGEL AB, Lund (SE); EXPLOATERINGS AB T.B.F., Uppsala (SE)

(72) Inventor: Jerker PORATH, BROMMA (SE)

(21) Appl. No.: 14/720,992

(22) Filed: May 26, 2015

Related U.S. Application Data
(63) Continuation of application No. PCT/SE2013/051410, filed on Nov. 28, 2013.

(30) Foreign Application Priority Data
Nov. 29, 2012 (SE) 1251351-1

(51) Int. Cl. B01J 20/22; C07C 37/70; C02F 1/28; B01J 20/24; B01D 15/36; C08B 37/00; C07C 7/12

(52) U.S. Cl. CPC B01J 20/22; C08B 37/0039; C07C 37/70; C07C 7/12; B01J 20/24; B01D 15/368; C02F 1/285; C02F 2101/305

(57) ABSTRACT

Adsorbent conjugate comprising a hydropolymer component covalently bound to a polyamine component, wherein the hydropolymer component is a substantially water-insoluble polysaccharide crosslinked with 2-hydroxypropylene moieties, the hydropolymer component forms a three-dimensional matrix in which the polyamine component is distributed, and wherein the polyamine component is a polyamine exhibiting terminal hydroxyl moieties, and the number of hydroxyl moieties is in excess of the number of amino groups. A column comprising the same and methods for its use.

### TECHNICAL FIELD

The present description relates generally to adsorbents and adsorbent materials, and in particular hydropolymer matrices with modified properties and increased binding capacity, suitable for adsorbing metals and aromatic compounds, e.g. different environmental pollutants, from aqueous solutions.

### BACKGROUND ART

Adsorbents are solids, frequently used in particulate form, capable of binding (adsorbing) compounds present in a liquid or gas phase surrounding them. Adsorbents can be made from inorganic or organic, synthetic or naturally occurring materials. Synthetic adsorbents frequently comprise an inert matrix, for example a polymer, to which active groups or ligands, have been attached.

Solid, particulate adsorbents have an advantage in that they can be packed in columns and subjected to considerable pressure and flow. This is not possible when for example dendritic, macromolecular adsorbents are used.

The adsorption capacity and specificity can be tailored by choosing specific ligands, for example creating an adsorption specificity drawn to specific groups of compounds, or in the extreme, to specific compounds. In cases where the matrix is a hydropolymer derivative having adsorption capabilities in itself, these can be enhanced, modified or supplemented with suitable ligands.

In U.S. Pat. No. 6,339,039 Jerker Porath and Bo Eriksson describe a water swellable adsorbent based on a water non-soluble support matrix which is cross-linked with polymers. An organic polymer or a combination of such, e.g. polysaccharides such as agar, cellulose, or starch, proteins and components of proteins and polysaccharides, is used as the support matrix. This support matrix is substituted with a first soluble polymer material chemically bound to said matrix, whereupon additional polymer materials are built into the primary synthesized support matrix polymer complex through different kinds of cross-links whereby optionally the support matrix is present in the form of an acid- and base-stable residue.

U.S. Pat. No. 8,097,165 (Caroline Mabille et al., Rhodia UK Ltd.) discloses that modified and insoluble starches can be utilized for eliminating natural organic substances/contaminants from liquids and in particular from liquids used for food applications, such as drinking water, beverages, fruit juices or syrups, as well as natural water, industrial process water, or wastewater. U.S. Pat. No. 8,097,165 describes in particular the introduction of cationic or cationizable groups, wherein “cationizable” means that these groups can be made cationic as a function of the pH of the medium.

WO 97/29825 (Rolf Berglund et al., Pharmacia Biotech AB) describes an anion exchanger which exhibits ligands, which (i) contain a primary, secondary or tertiary amino group, and (ii) are covalently bound to an organic polymer matrix. Further, there is a hydroxyl group or a primary, secondary or tertiary amino group on a carbon atom at a distance of 2 or 3 atoms away from the amino nitrogen in the ligand. WO 97/29825 focuses on the separation of nucleic acids. The following examples of matrixes are mentioned: polyhydroxy polymers based on polysaccharides, such as agarose, dextran, cellulose, starch, pullulane etc., and purely synthetic polymers, such as polyacrylamide, polymethacrylamide, poly(hydroxyalkyl vinyl ethers), poly(hydroxyalkyl acrylate) and corresponding polymethacrylate, polyvinyl alcohol, and polymers based on styrene and divinylbenzene (DVB), and copolymers where corresponding monomers are included.

As set out for example by G. Crini in “Recent developments in polysaccharide-based materials used as adsorbents in wastewater treatment” (Prog Polym Sci, 30 (2005) 38-70) there is a growing interest in the synthesis of new low-cost adsorbents for use in wastewater treatment. The review article presents recent developments in the synthesis of adsorbents containing polysaccharides, in particular modified biopolymers derived from chitin, chitosan, starch and cyclodextrin.

Dendrimer-based adsorbents, as shown for example in Chen, P. et al. “A Tris-Dendrimer for Hosting Diverse Chemical Species” (in J. Phys. Chem. C, 2011, vol. 115, s. 12789-12796) represent a different approach. These have insufficient mechanical strength and are therefore not suitable for packed columns, where high pressure and/or flow is/are desired.

The development of new, improved synthetic or semi-synthetic adsorbents remains a challenge, in particular as many properties need to be considered: physical strength, chemical stability, regeneration properties, adsorption capacity and specificity, as well as cost.

### SUMMARY

Therefore and according to a first aspect, there is provided an adsorbent conjugate comprising a hydropolymer component (A) covalently bound to a polyamine component (B), wherein the hydropolymer component (A) is a substantially water-insoluble polysaccharide crosslinked with 2-hydroxypropylene moieties, and the hydropolymer component (A) forms a three-dimensional matrix in which the polyamine component (B) is distributed; wherein the polyamine component (B) is a polyamine exhibiting terminal hydroxyl moieties, and the number of hydroxyl moieties is in excess of the number of amino groups i.e. OH/N>1. According to a preferred version the ratio OH/N≧2 or more preferably 3 or higher.

According to one version, the hydropolymer component (A) in said adsorbent conjugate is chosen from agar and agarose.

According to one version, the hydropolymer component (A) is present in the form of particles or fiber bundles. According to a preferred version said particles or fiber bundles have a diameter of 1-1000 μm, for example about 5 to about 500 μm.

According to a preferred version, freely combinable with the above versions, said polyamine component (B) exhibits a network structure of amino moieties separated by 2-hydroxy propylene or ethylene bridges.

According to one version, freely combinable with the above versions, said polyamine component (B) is substituted with tris(hydroxymethyl) aminomethane (TRIS) and optionally other ligands exhibiting terminal hydroxyl moieties. The TRIS substitution can be repeated as many times as necessary, until the desired density of available hydroxyl moieties has been reached.

According to a preferred version, freely combinable with the above versions, the polyamine component (B) consists substantially of tris (hydroxymethyl) aminomethane crosslinked with 2-hydroxy propylene bridges.

According to one version, freely combinable with the above versions, the polyamine component (B) is chosen from oligo(ethyleneimine) and poly(ethyleneimine).

According to one version, freely combinable with the above versions, the polyamine component (B) is a polyamine crosslinked by a bridge compound comprising at least five carbon atoms and one or more of O, N or S.

According to the above version, said bridge compound has a structure chosen from

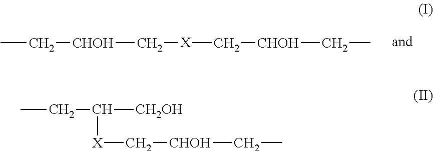

or multiples thereof, wherein X is chosen from O, N, or S.

According to the above version, said bridge compound is preferably extended by one of more moieties chosen from a diamine, a dithiol, and 2-hydroxy propylene.

Further, according to the above version, said bridge compound has the structure

—CH₂—CHOH—CH₂—Y—CH₂—CHOH—CH₂— (III)

wherein
Y is —[(NH—CH₂—CH₂—)ₚ]ₙ—NH—, and
p≧2, n≧2.

According to one version, said diamine is a cyclic diamine.

Further, according to the above version, Y is preferably —NH—CO—NH—.

According to a second aspect, there is provided a sorbent column comprising an adsorbent conjugate according to the first aspect and any versions thereof.

In chemical compounds, so called π-bonds (pi-bonds) result from an overlap of atomic orbitals, forming diffuse bonds, termed π-bonds in contrast to ζ-bonds (sigma-bonds). Together these form a strong double bond. Electrons participating in π-bonds are termed π-electrons.

According to a third aspect, there is provided a method for the removal of π-electron rich compounds from aqueous solutions using an adsorbent conjugate and/or a sorbent column according to the first and second aspects and any versions thereof.

There is also provided a method for the removal of aromatic compounds from aqueous solutions using an adsorbent conjugate and/or a sorbent column according to the first and second aspects and any versions thereof.

There is also provided a method for removal of alkenes and alkynes from aqueous solutions using an adsorbent conjugate and/or a sorbent column according to the first and second aspects and any versions thereof.

There is also provided a method for the purification of effluent streams, in particular for the removal of drug residues in waste water, using an adsorbent conjugate and/or a sorbent column according to the first and second aspects and any versions thereof.

In general, there is provided a method for the purification of effluent streams, for example for the removal of drug residues from process effluents in the pharmaceutical industry, municipal and industrial waste waters, ground water and surface water, as well as from drinking water using an adsorbent conjugate and/or a sorbent column according to the first and second aspects and any versions thereof. The adsorbent conjugate has been shown to be useful for the removal of aromatic compounds of both small and large molecular size, as well as metals, exemplified by copper. This has potential utility in the purification of effluents, recovery of metals in industrial processes, as well as in the purification of drinking water, food stuffs and the like. The principle of superhydroxylation makes it possible to tailor adsorbents for specific uses.

Another aspect concerns the production of an adsorbent material, wherein the polysaccharide to be used as matrix, for example agar, is concentrated to a physically suitable form, preferably spherical fiber bundles or beads, permitting packing of the matrix in beds exhibiting high flow rates to passing fluids. In the alternative, a commercially available or naturally occurring polysaccharide is used as available, with no or only little modification, if it already exhibits the desired properties.

While many particles used in adsorption columns are spherical, this is not the only possible shape. Particle parameters include particle size, shape, porosity and physical hardness. The particle parameters are chosen so, that the necessary flow and adsorption is achieved in each particular case. A skilled person understands that different columns or particle beds pose different challenges, and that for example the height and diameter of the column need to be considered when choosing particle size and hardness.

The beaded polysaccharide is then treated with a halohydrin reagent or a halohydrin generating reagent, for example allylbromide and bromine, in two or more sequential operations to increase the hardness of the particle. In the alternative, a commercially available or naturally occurring polysaccharide is used as available, with no or only little modification, if it already exhibits the desired hardness.

The activated beaded and activated polysaccharide is then substituted by hydroxyl, amino or thiol groups. The resulting particles are then activated using a substance comprising reactive halogen groups (e.g. bromohydrin) or methylol groups and are then converted to superhydroxylated adsorbents by substitution with (trishydroxy methyl) amino methane (TRIS) or hydroxyl generating reagents (e.g. bifunctional reagents such as glycidol or ethylene oxide).

The above steps are repeated as many times as necessary to produce the desired adsorbent exhibiting hydroxyl moieties.

While the most preferred version of the adsorption conjugate and methods of its use comprise a conjugate having both polyamine and TRIS-substitutions, it is conceived that a very useful adsorbents can be obtain by multiple TRIS-substitution of the polysaccharide directly, without preceding polyamine substitution. The TRIS-substitution will result in a denser distribution of the hydroxyl moieties on the surface of the adsorption conjugate, which is turn may be advantageous for the adsorption of smaller molecules. Conversely, a polyamine substitution is believed to result in a sparser distribution of hydroxyl moieties, advantageous for the adsorption of larger molecules. By combining different degrees of polyamine and TRIS substitution, the adsorption properties can tailored as desired.

### SHORT DESCRIPTION OF THE DRAWINGS

It will be readily apparent that other variations and modifications are possible in accordance with the following detailed description, which should be read in conjunction with the accompanying drawings, in which

FIG. 1 shows schematically how a polysaccharide particle consists of a tangled ball of polysaccharide fiber bundles.

FIG. 2 is a cross section of fiber bundles in a particle such as that shown in FIG. 1, illustrating how hydroxyl moieties are exposed in the cavities and interstices formed in the particle.

FIG. 3 is a detail view showing how each fiber bundle in turn consists of a multitude of cross-linked polysaccharide molecules, and how the fiber bundle exposes hydroxyl moieties at its surface.

### DETAILED DESCRIPTION

Before the aspects and versions are described, it is to be understood that the terminology employed herein is used for the purpose of describing particular aspects and versions only and is not intended to be limiting, since the scope will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of +/−2% of the given value, preferably +/−5%, and most preferably +/−10% of the numeric values, where applicable.

The term “hydropolymer” is used to describe a bi-phasic structure including a water absorbing resin, forming a three-dimensional matrix, and a liquid component distributed in said matrix. It is contemplated that the adsorption takes place in the interphase between the solid and liquid phase, and that the large surface area of the water absorbing resin potentiates the adsorption capacity of the hydropolymer.

The term “superhydroxylated” is used to describe that the adsorbent matrix presents a high concentration of hydroxyl groups. Without wishing to be bound to any particular theory, it is however contemplated that the density and distribution of hydroxyl groups or moieties on the adsorbent material significantly influences the adsorption capacity and specificity of the adsorbent. ***

According to a general version, the cross-linked hydropolymer is chosen from agar, agarose, cellulose, hemicelluloses, starches, chitin, chitosan, and bacterial polysaccharides, including e.g. pectin and dextran. Preferably agar, agar-based or agar-containing materials are chosen, as these exhibit high mechanical strength, which allows high flow, and tolerate large variations in pH. In particular the latter is of significance, as a high pH tolerance allows rapid and efficient regeneration of the adsorbent.

According to one version, freely combinable with other versions disclosed herein, the polysaccharide is derivatized to a cross-linked polyamine-polysaccharide conjugate and used as the supporting matrix for particular adsorbents. The polyamine component is less hydrophilic than the polysaccharide, however the amine component is more easily alkylated and that may compensate for the decrease in hydrophilicity if alkylation is made by a hydroxyl producing reagent.

The matrix is preferably a particulate matrix, and the particle size is, depending on the intended use in the interval of about 1 to about 2000 μm, for example 1 to about 1000 μm and preferably in the interval of about 5 to about 500 μm, or in the interval of about 50 to about 200 μm. A person skilled in the art knows that column performance is affected both by column parameters (such as the diameter and length), particle parameters (notably particle size and shape), the type of eluent (especially its viscosity), and flow rate or average linear velocity. The performance is also affected by the compound to be adsorbed, and its retention.

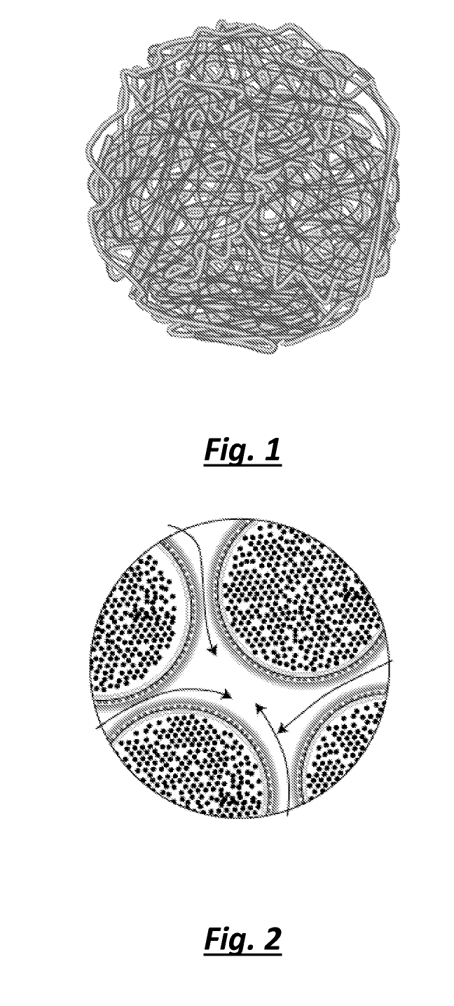

FIG. 1 shows schematically a particle formed by entangled fiber bundles, illustrating for example an agarose particle or bead, as frequently referred to in this description.

FIG. 2 shows partial cross sections of four fiber bundles comprised in a particle as shown in FIG. 1. The black dots illustrate the multitude of cross-linked agarose molecules forming each fiber bundle, and the shaded area indicates the surface which exposes hydroxyl moieties. The arrows indicate how a liquid enters the interstices between the fiber bundles during substitution of the adsorbent conjugate, or during use, when a liquid to be purified is passed through a bed of particles.

FIGS. 1 and 2 together illustrate the high surface area available in a particle of this type. It is apparent that a particle of this type has an extremely large surface area.

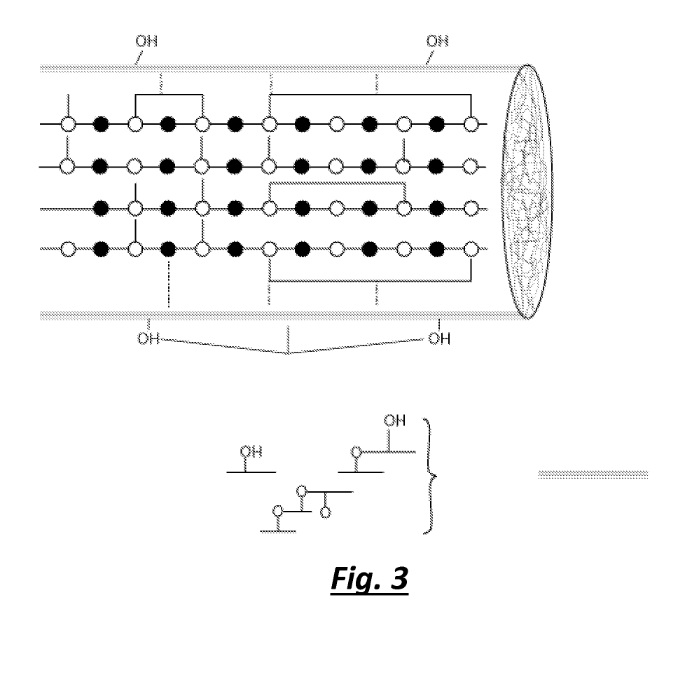

FIG. 3 in turn schematically illustrates how each fiber bundle in turn consists of a multitude of cross-linked polysaccharide molecules, and how the fiber bundle exposes hydroxyl moieties at its surface.

Different sizes and qualities of beads can be obtained from commercial sources. Agarose beads are available from inter alia Inovata AB, Sweden; Merck Millipore, Germany; Agarose Bead Technologies Inc., USA; Vector Laboratories Ltd., UK, etc. The advantage of agarose is that it forms a highly porous and physically stable matrix. Agarose based matrices have been successfully used over decades in both research and industrial applications.

Spherical cellulose beads exhibiting high chemical stability and high mechanical strength are supplied inter alia by JNC Corporation, Tokyo, Japan (the Cellufine® product line.

Dextran beads are available for example as the Cytodex® product line, as beads of a size in the interval of 60 to 87 μm (Sigma-Aldrich, www.sigmaaldrich.com).

Pure starch, as well as different mixtures, such as starch mixed with alginate, can be used for the production of beads. These are currently used as spherical seed cores for drug layering & film coating. They also find use in the manufacture of granules/beadlets with sustained/controlled release, taste masking & other special properties (Umang Pharmatech PVT.LTD., India).

Alginate and chitin beads are available for example from New England Biolabs, USA. Chitosan is a polycation that can be cross-linked with multivalent anions, and can be used to prepare beads.

The manufacture of beads of different sizes is also well known to a person skilled in the art. The substrate (e.g. cellulose) is dissolved in a chaotropic solvent, formed into droplets and immersed into a non-solvent capable of solvent interchange with the first solvent, to form generally spherical porous beads of narrow particle size distribution. It is for example well known that pectin solutions can be dropped into concentrated calcium chloride solutions, whereby the pectin gels instantaneously, and form beads.

Most preferably the matrix, e.g. the particles, is/are chosen from agar and agarose. These form substantially non-elastic particles which are mechanically stable and withstand high pressure. This makes them suitable for use in columns, where they enable high flow without noticeable compression.

One method for the manufacture of an agarose separation gel is disclosed in the international patent application published as WO 2008/136742 (Method for the manufacture of agarose gels, inventor: Goran Lindgren) included herein by reference. The method disclosed in WO 2008/136742 comprises the steps of:

i) providing a solution of agar, and
ii) one, two or more intermediate steps which each comprises a desulphating reaction thereby transforming agar to a product having a degree of substitution of sulphate groups that is at most 75% of the degree of substitution of sulphate groups in native agar,
iii) gelling the dissolved agar prior to step (ii) and/or securing that the desulphated agar is in gel form at least after one or more of the intermediate steps of step (ii), and imperatively after step (iii).

The resulting product is an agarose separation gel that exhibits on the one hand a plurality of methoxy groups each of which are at the same position as in native agar and with a degree of substitution in the range of 1-100% of the degree of substitution of native agar, and on the other hand sulphate groups with a degree of substitution which is <75% of the degree of substitution for sulphate groups in native agar.

A polysaccharide crosslinked separation material in particulate form exhibiting good mechanical strength can also be manufactured as disclosed in EP 0132244 (Separation material and its preparation, inventors: Göran Lindgren, Mats Carlsson, Per-Ake Pernemalm) incorporated herein by reference.

The superhydroxylated hydropolymers according to one version exhibit two types of hydrogen bonds:

(a) Hydrogen bonds between the adsorbent and oxygen containing substances having at least one free electron couple, such as alcohols, phenols, ethers, ketones, and carboxylic acids. These can be schematically illustrated as follows:

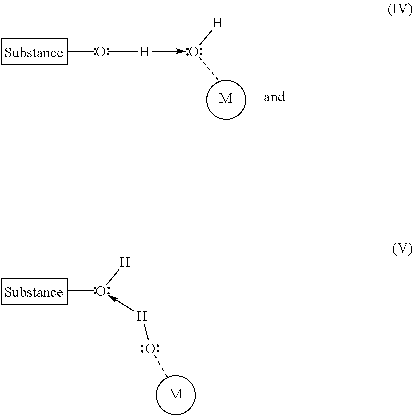

(b) Hydrogen bonds between the adsorbed substance and an unsaturated group with double bonds having π-electrons:

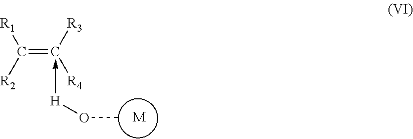

or more efficiently to the π-electron system in an aromatic or hetero aromatic moiety:

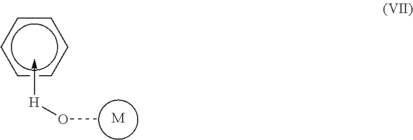

According to a general aspect, each ligand presents a local, high concentration of OH-moieties. These OH-groups can further be substituted by

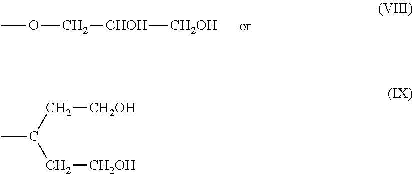

which further enhance the hydrogen bond related adsorption.

The adsorption can be further increased by adding a trihydroxymethyl moiety:

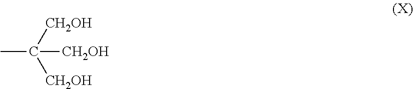

This moiety can be synthesized by transformation of acetaldehyde with formalin under basic conditions. The OH-moieties can also be introduced through alkylation followed by bromide treatment and coupling of allyl ether and TRIS (trihydroxy methyl aminomethane) to synthesize the following group:

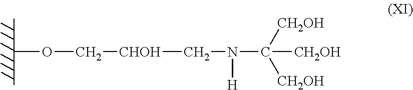

The allyl bromide treatment, subsequent bromation in water, and TRIS coupling is repeated as desired. By engaging hydroxyl moieties in the crosslinkter, a multi-dimensional network is formed, comprising amino moieties substitueted with 2-hydroxy propylene bridges and having TRIS distributed within the network.

Further, the hydrogen bound to the nitrogen can then be selectively substituted, introducing additional OH-moieties:

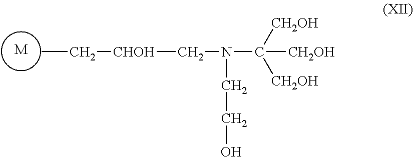

For example, following an addition of Cl—CH₂—CH₂OH, the following structure is achieved:

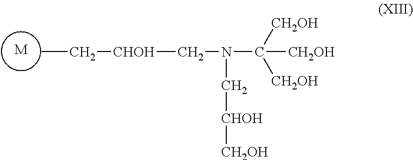

This can be subjected to further treatment and thus given an increased affinity and/or selective affinity for example by adding alkyl groups to impart hydrophobicity, dinitrophenyl to impart electron acceptor affinity, metal chelating groups to impart metal affinity etc.

The nitrogen can be derivatized further, yielding structures such as:

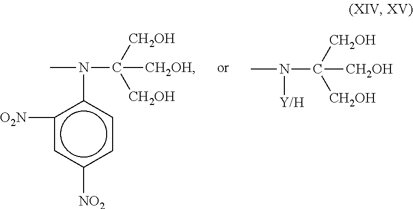

where Y is chosen from alkyl, allyl, or for example —CH₂—CHO—CH—Z, wherein Z can be SO₃H etc.

The ligand is also preferably selectively substituted with TRIS to gain high, local hydroxyl concentration:

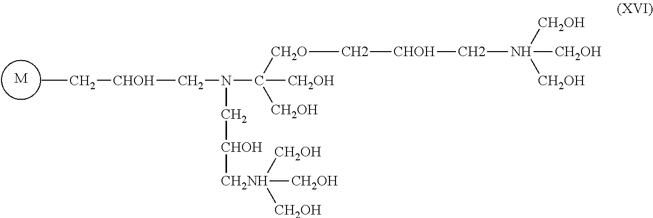

In the above formula (XVI), only one of the CH₂OH moieties is extended, but preferably two or three are extended in a similar fashion. In this manner, adsorbents with increasing adsorption capacity are produced.

According to a further version, the polyamine is a linear polyamine, for example tetra ethylene pentamine. This can be attached to the hydropolymer component forming the support matrix:

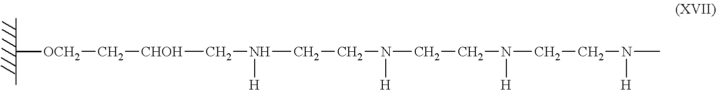

This is then activated, and substituted with TRIS to give the following structure:

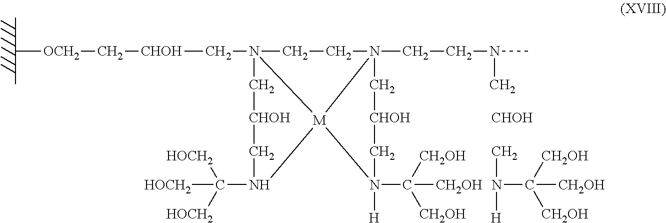

Note that the nitrogen atoms are now in position for forming metal ion complexes (the position of a metal indicated with “M”).

The structures according to an aspect can be summarized as a structure wherein the central atom, here nitrogen, is surrounded by four OH-moieties, wherein three are primary, and wherein all OH-moieties are present at a distance from the central atom corresponding to a carbon chain of a maximum of two carbon atoms (—C—C—).

The high concentration of OH-moieties on the adsorbent matrix can also be achieved using other central atoms, such as oxygen, carbon or sulphur:

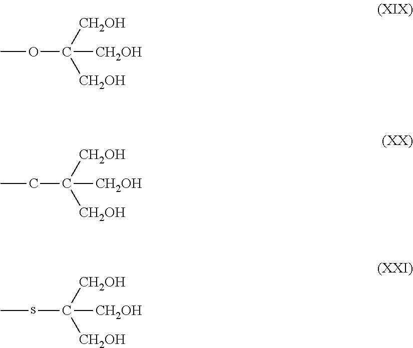

These ligands can be termed “group ligands” or “cluster ligands” and by varying the amount of hydrogen binding moieties on each ligand, and the concentration of ligands on the matrix, the adsorption capacity and specificity of the adsorbent can be tailored as desired.

These compounds can be synthesized from aldehyde moieties —CH₂CHO or precursors generating this group using formaldehyde or other suitable reagents, for example glycidol (oxiranylmethanol) and according to methods well known to persons skilled in the art.

The OH moieties as such have a weak affinity for organic compounds, such as alcohols, phenols, ethers and ketones. In case the affinity is insufficient, and efficient adsorption cannot be achieved in aqueous solutions, a salt can be added, for example 1 M alkali sulphate or phosphate.

It has however now been shown that adsorption is significant also in the absence of salt, as the hydrogen adsorption resides in the hydropolymer derivative according to different versions. Without wishing to be bound to any particular theory, this is possibly explained by the cooperative effect of the hydroxyl groups present in high local concentrations on the absorbent matrix. This makes the adsorbent useful for the removal of organic compounds in water, for example in the purification of effluents from chemical or pharmaceutical manufacturing, or for the removal of organic contaminants such as drug residues in drinking water. Surprisingly, aromatic compounds, including aromatic compounds with a low degree of substitution, are adsorbed without the concurrent use of water-structuring salts, such as alkali sulphates and alkali phosphates.

Further, the cross-linked hydropolymer matrix has an advantage in that it is physically and chemically highly stable due to the combination of the cross-linking and the additional hydrophilization.

The increased physical stability also has an important advantage in that a column packed with an adsorbent as disclosed herein, can be subjected to higher flow and pressure than conventional adsorbents with similar adsorption capacity.

The improved chemical stability has been shown inter alia by subjecting adsorbents (produced as in the attached examples) to strong acids, for example by immersing the adsorbent in 30% sulphuric acid for a week at room temperature. No visual changes were recorded. Similar tests, using strong oxidizing agents such as 0.5 M potassium iodate, confirmed the exceptional stability of the adsorbent. Based on preliminary experiments, it is estimated that these adsorbents can withstand repeated regeneration for a number of cycles, far higher than what is possible using a conventional adsorbent.

The improved stability also makes it possible to regenerate the adsorbent more rapidly, for example using a strong alkali.

Additionally, the gel character of the adsorbent results in faster adsorption and desorption, and hydrophilic adsorbent, like that disclosed herein, has the added advantage of being possible to use directly in the aqueous medium, without the need of pre-treatment steps potentially involving other harmful chemicals, such as a solvent extraction step or the like.

### EXAMPLES

### Example 1

### Chemical Modification of Novarose™

240 g tris(hydroxymethyl) aminomethane (TRIS) was dissolved in 600 ml water. To this solution 607 g of bromohydrin activated Novarose™ SE 1000/40 was added. Novarose SE 1000/40 is a spherical, highly cross-linked agarose, available from Inovata AB, Bromma, Sweden (http://www.inovata.se/sec/).

The reaction mixture was stirred over night. The gel was than washed thoroughly on a glass filter during suction.

### Example 2

### Chemical Modification of Novarose™—Continued

252 g allyl bromide, 198 g water and 615 g NaOH was added to 580 g of the product obtained in Example 1. The reaction mixture was stirred over night and then washed on a glass filter funnel. To the resulting gel, 500 ml water was added and bromine water was added until all allyl groups had reacted. The gel was thoroughly washed on a glass filter funnel. Yield: 581 g.

222 g tris(hydroxymethyl)aminomethane (TRIS) was dissolved in 560 ml water. To this solution, 561 g of the gel above was added. The reaction mixture was stirred over night and then washed on a glass filter funnel.

### Example 3

### Chemical Modification of Novarose™—Continued

400 g of the product obtained in Example 2 was treated with allyl bromide and TRIS in the same way, forming a superhydroxylated particulate adsorbent. Yield 400 g.

### Example 4

### Removal of Aromatic Compounds from Water (2-Napthol)

In a laboratory experiment, a dilute solution of 2-naphtol in water was used in a simulation of the cleaning of contaminated water. A high pressure liquid chromatography system, comprising a pump (Pharmacia LKB 2248) connected to a UV spectrometer and detector (LKB 2510 Uvicord SD (276 nm)) and a chromatogram recorder (LKB 2210) and LC controller (LKB 2252) was assembled. The gel obtained in Example 3 was packed in a 50×8 mm glass column and connected to the above HPLC system using ¼″-28 fittings with 1/16″ tubings. A solution of 2-naphtol in water (0.5 mg/ml) was run through the column with a flow rate of 0.3 ml/min.

A breakthrough was noted after 258 ml which corresponds to 129 mg 2-naphtol. This equals an adsorption capacity of 51.6 mg/ml gel. The results show that the adsorbent was capable of binding in excess of 50 mg 2-naphtol/ml adsorbent gel. Calculated on the dry weight of the gel, this corresponds to a significant adsorption.

### Example 5

### Removal of Aromatic Compounds from Water (Bisphenol A)

1 gram of bisphenol A was dissolved in 10 l of water under stirring over night. This solution was injected and run on an isocratic (100% H₂O) system, through a 50×8 mm column packed with the gel obtained in Example 3 with a flow rate of 1 ml/min. The chromatography system was assembled as disclosed in Example 4.

A breakthrough was noted after 795 ml which corresponds to an adsorption of 79.5 mg bisphenol A. Thus, the adsorbent had a binding capacity of 31.8 mg bisphenol A per ml of gel. Calculated on the dry weight of the gel, this corresponds to a significant adsorption.

### Example 6

### Comparative Example Showing Effect of Repeated TRIS Substitution

Three 2.5 ml 5 cm columns were prepared, each packed with one of Novarose 1000/40, the gel obtained in Example 1, and the gel obtained in Example 2. Benzyl alcohol dissolved in H₂O was injected and run on an isocratic (100% H₂O) system. The chromatography system was assembled as disclosed in Example 4, and operated as disclosed in Table 1.

TABLE 1
Experimental set-up

| Injection: | 50 | μL benzyl alcohol |
|---|---|---|
| Concentration: | 35 | mg/mL |
| Range: | 0.08 | |
| Chart speed: | 5 | mm/min |
| Flow: | 1 | mL/min |

The results (Table 2) clearly show the improved adsorption and affinity compared to untreated Novarose™ as well as the increased affinity when the amount of ether- and hydroxyl groups increases.

TABLE 2
Results

| Sample: | Result (mm): |
|---|---|
| Novarose ™ SE 1000/40 | 32 |
| Gel obtained in Example 1 | 57 |
| Gel obtained in Example 2 | 79 |

It can be seen from the results in Table 2, that the gel obtained in Example 1 exhibited a retention capacity which was 78% higher than that of the control (untreated Novarose™). The gel obtained in Example 2 exhibited a retention capacity 147% higher than that of the control, and an improvement of 39% compared to the gel obtained in Example 1.

### Example 7

### Synthesis of PEI Substituted Gel

A slurry of 1000 ml Novarose® (an un-derivitazed cross-linked agar gel, particle size 200 μm) in 800 ml water and 200 ml 6.2 M NaOH was reacted with 76 g of allyl bromide over night under stirring. After washing with water the gel was treated with bromine water.

1000 ml solution containing 200 g polyethyleneimine (50% in water) was added and stirred over night at room temperature. The mixture was thoroughly washed with water.

### Example 8

### Removal of Cu²⁺ in Water

A 2.5 ml Novaline glass column (50×8 mm) was packed with the gel obtained in Example 9 and tested with 0.1 M Cu(NO₃)₂. The total uptake was 12 mg/ml.

### Example 9

### Synthesis of PEI and TRIS Substituted Gel

550 g water and 110 g of allyl bromide was added to a sample of 550 g PEI-gel obtained in Example 7. The pH of the slurry was 9.7 due the PEI gel. The mixture was stirred over night. The gel was washed thoroughly with water on a glass funnel and placed in a beaker. Bromine water was added and the gel was subsequently washed again on a glass funnel. To the activated gel 110 g of TRIS was added and reacted under stirring over night. Some aggregates were noted after washing.

### Example 10

### Removal of Tetracycline

A 2.5 ml Novaline glass column (50×8 mm) was packed with the gel in Example 9. A solution of 200 mg of tetracycline (Sigma-Aldrich Co., catalogue no. 87128) in 1000 ml water was prepared. The column was connected to a HPLC system (LKB HPLC pump 2248, 2252 LC Controller, 2151 Variable wavelength monitor, Reodyne Injector and Kipp & Zonen recorder, Kipp & Zonen B.V., The Netherlands) and the tetracycline solution was pumped through column at a flow rate of 1.25 ml/min (0.25 mg tetracycline/ml). A breakthrough after 240 ml shows an uptake of 24 mg/ml gel.

The saturated gel was washed by passing ethanol through the column with until all tetracycline was desorbed and then with water. The experiment was repeated with the same result. It is a considerable advantage of gel that it can be regenerated using ethanol.

### Example 10

### Removal of Toluene

A saturated water solution of toluene (0.06%) was run through an 8×25 ml column packed with the gel in Example 3 on the HPLC-system at the wavelength 260 nm.

A breakthrough after 35 ml of the saturated solution gives an uptake of about 9 mg toluene per ml gel.

1. An adsorbent conjugate comprising a hydropolymer component covalently bound to a polyamine component, wherein
the hydropolymer component is a substantially water-insoluble polysaccharide crosslinked with 2-hydroxypropylene moieties,
the hydropolymer component forms a three-dimensional matrix in which the polyamine component is distributed,
wherein
the polyamine component is a polyamine exhibiting terminal hydroxyl moieties and amino groups,
wherein the hydroxyl moieties are in excess of the number of amino groups.

2. The adsorbent conjugate according to claim 1, wherein the number of hydroxyl moieties/number of amino groups ≧2.

3. The adsorbent conjugate according to claim 1, wherein the hydropolymer component is chosen from agar and agarose.

4. The adsorbent conjugate according to claim 1, wherein the hydropolymer component is present as particles or fiber bundles.

5. The adsorbent conjugate according to claim 4, wherein said particles or fiber bundles have a diameter in an interval of about 1 to about 2000 μm.

6. The adsorbent conjugate according to claim 1, wherein the polyamine component exhibits a network structure of amino moieties separated by 2-hydroxy propylene or ethylene bridges.

7. The adsorbent conjugate according to claim 1, wherein the polyamine component is substituted with tris(hydroxymethyl) aminomethane (TRIS) and optionally other ligands exhibiting terminal hydroxyl moieties.

8. The adsorbent conjugate according to claim 1, wherein the polyamine component consists substantially of tris(hydroxymethyl) aminomethane crosslinked with 2-hydroxy propylene bridges.

9. The adsorbent conjugate according to claim 1, wherein the polyamine component is chosen from oligo(ethyleneimine) and poly(ethyleneimine).

10. The adsorbent conjugate according to claim 1, wherein the polyamine component is a polyamine crosslinked by a bridge compound comprising at least five carbon atoms and one or more of O, N or S.

11. The adsorbent conjugate according to claim 10, wherein said bridge compound has a structure chosen from 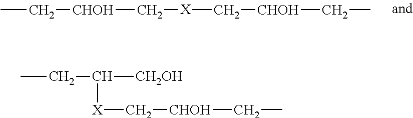
or multiples thereof, wherein X is chosen from O, N, or S.

12. The adsorbent conjugate according to claim 10, wherein said bridge compound is extended by one or more moieties chosen from a diamine, a dithiol, and 2-hydroxy propylene.

13. The adsorbent conjugate according to claim 10, wherein said bridge compound has the structure
—CH₂—CHOH—CH₂—Y—CH₂—CHOH—CH₂—
wherein
Y is —[(NH—CH₂—CH₂—)ₚ]ₙ—NH—, and
p≧2, n≧2.

14. The adsorbent conjugate according to claim 12, wherein said diamine is a cyclic diamine.

15. The adsorbent conjugate according to claim 13, wherein Y is —NH—CO—NH—.

16. A sorbent column comprising an adsorbent conjugate according to claim 1.

17. A method for removal of π-electron rich compounds from aqueous solutions using an adsorbent conjugate according to claim 1.

18. A method for removal of aromatic compounds from aqueous solutions using an adsorbent conjugate according to claim 1.

19. A method for removal of alkenes and alkynes from aqueous solutions using an adsorbent conjugate according to claim 1.

20. A method for the purification of effluent streams, in particular for the removal of drug residues in waste water, using an adsorbent conjugate according to claim 1.

21. A method for the purification of drinking water using an adsorbent conjugate according to claim 1.

22. A method for removal of π-electron rich compounds from aqueous solutions using an adsorbent conjugate according to a column according to claim 16.

23. A method for removal of aromatic compounds from aqueous solutions using an adsorbent conjugate according to a column according to claim 16.

24. A method for removal of alkenes and alkynes from aqueous solutions using an adsorbent conjugate according to a column according to claim 16.

25. A method for the purification of effluent streams, in particular for the removal of drug residues in waste water, using an adsorbent conjugate according to a column according to claim 16.

26. A method for the purification of drinking water using an adsorbent conjugate according to a column according to claim 16.
